(19) United States
(12) Patent Application Publication
(10) Pub. No.: US 2010/0227381 A1
(43) Pub. Date: Sep. 9, 2010

(54) ENHANCED BIODEGRADATION OF NON-AQUEOUS PHASE LIQUIDS USING SURFACTANT ENHANCED IN-SITU CHEMICAL OXIDATION

(75) Inventors: George E. Hoag, Storrs, CT (US); John Collins, Amston, CT (US)

Correspondence Address: VENABLE LLP, P.O. BOX 34385, WASHINGTON, DC, 20043-9998, US

(73) Assignee: VERUTEK TECHNOLOGIES, INC., Bloomfield, CT (US)

(21) Appl. No.: 12/670,373

(22) PCT Filed: Jul. 23, 2008
(86) PCT No.: PCT/US08/08905

§ 371 Date: Jan. 22, 2010

Related U.S. Application Data
(60) Provisional application No. 60/935,033, filed on Jul. 23, 2007.

(51) Int. Cl. A62D 3/02; C02F 1/72; C02F 1/70; C02F 3/30; B09C 1/08; B09C 1/10; G01N 33/24

(52) U.S. Cl. 435/262.5; 210/631; 210/630; 210/620; 210/610; 210/170.07; 210/96.1; 210/614; 436/183

(57) ABSTRACT

A method for in-situ reduction of contaminants in soil that uses chemical oxidation and biodegradation.

### BACKGROUND

The present invention relates to methods and compositions for remediating soil and groundwater. For example, the present invention relates to methods and compositions for removing contaminants from soil and groundwater in situ using surfactants or surfactant-cosolvent mixtures and oxidants to induce chemical oxidation and biodegradation.

Bioremediation can use organisms such as green plants, bacteria, fungi, or microorganisms to return an environment altered by contaminants to its original condition. Examples of bioremediation include attacking specific soil contaminants, such as chlorinated hydrocarbons, by bacteria, and cleaning up oil spills by the addition of fertilizers (such as nitrate and sulfate fertilisers) that facilitate the bacterial decomposition of crude oil. For a long time, phytoextraction has been used to desalinate agricultural land. Examples of bioremediation technologies include bioventing, landfarming, bioreactor, composting, bioaugmentation, rhizofiltration, and biostimulation.

### SUMMARY

A method according to the invention for reducing the concentration of a contaminant in a subsurface to a predetermined level can include the following. A primary oxidant and a surfactant and/or a cosolvent can be introduced into the subsurface. The surfactant can solubilize and/or desorb the contaminant. The primary oxidant can oxidize the solubilized contaminant in the subsurface to a biodegradable compound. A microorganism in the subsurface can biodegrade the biodegradable compound. The method can reduce the concentration of the contaminant in the subsurface to the predetermined level.

The method can include monitoring the subsurface for a quantity selected from the group consisting of contaminant concentration, oxidant concentration, surfactant concentration, cosolvent concentration, microorganism concentration, and combinations. The information obtained by monitoring can be used to adjust the amount of oxidant, surfactant, and/or cosolvent introduced into the subsurface to minimize the amount of contaminant in the subsurface.

The method can include establishing an oxidative zone in the subsurface with the primary oxidant in the vicinity of the locus of introduction of the primary oxidant. Aerobic microorganisms can proliferate in the oxidative zone and biodegrade the biodegradable compound and/or the contaminant. Oxidation of the solubilized contaminant and/or biodegradation of the biodegradable compound by the microorganism can consume the primary oxidant and can establish a reductive zone in the subsurface surrounding the oxidative zone. Anaerobic microorganisms can proliferate in the reductive zone and biodegrade the biodegradable compound and/or the contaminant.

A method according to the invention of designing a procedure for reducing the concentration of a contaminant at a site in a subsurface can include the following. A sample representative of the contaminated site of interest can be obtained. The sample can be tested with various concentrations of primary oxidant, surfactant, and/or cosolvent under various conditions of temperature, pressure, and/or flow rate. The rate of mobilization of the contaminant under the various concentrations and conditions can be determined. The rate of biodegradation of the contaminant under the various concentrations and conditions can be determined. An optimum set of concentrations and conditions for reducing the concentration of the contaminant at the site in the subsurface can be identified.

In an embodiment according to the invention, a system for reducing the concentration of a contaminant at a site in a subsurface can include an injection well, an injection fluid injection system fluidly connected to the injection well, and an injection fluid. The injection fluid can include a primary oxidant and a surfactant and/or a cosolvent. The system can operate to promote biodegradation of the contaminant.

The system can include a first pumping system and a second pumping system. The first pumping system can store a primary oxidant and a surfactant and/or a cosolvent, mix the primary oxidant and the surfactant and/or cosolvent in predetermined ratios, and inject the primary oxidant and the surfactant and/or cosolvent at a first injection point into an oxidation zone of the subsurface at a predetermined rate. The second pumping system can store a reducing agent and inject the reducing agent at a second injection point into a reducing zone of the subsurface at a predetermined rate. A monitoring device can determine the concentration and/or spatial distribution in the subsurface of a quantity selected from the group consisting of contaminant, oxidant, surfactant, cosolvent, microorganisms, and combinations. The monitoring device can adjust the ratios of mixing the primary oxidant and the surfactant and/or cosolvent, the rate of injection of the primary oxidant and the surfactant and/or cosolvent, and the rate of injection of the reducing agent, so as to maximize biodegradation by the microorganisms, minimize the concentration of the contaminant, minimize the time required to reduce the contaminant to a predetermined level, and/or minimize the amount of primary oxidant, surfactant, cosolvent, and/or reducing agent used.

### BRIEF DESCRIPTION OF THE FIGURES

FIG. 1A illustrates processes in a combined S-ISCO™—reductive biodegradation approach for remediating a NAPL contaminated site.

FIG. 1B illustrates the process of oxidation of contaminant by S-ISCO.

FIG. 1C illustrates the process of biodegradation of contaminant.

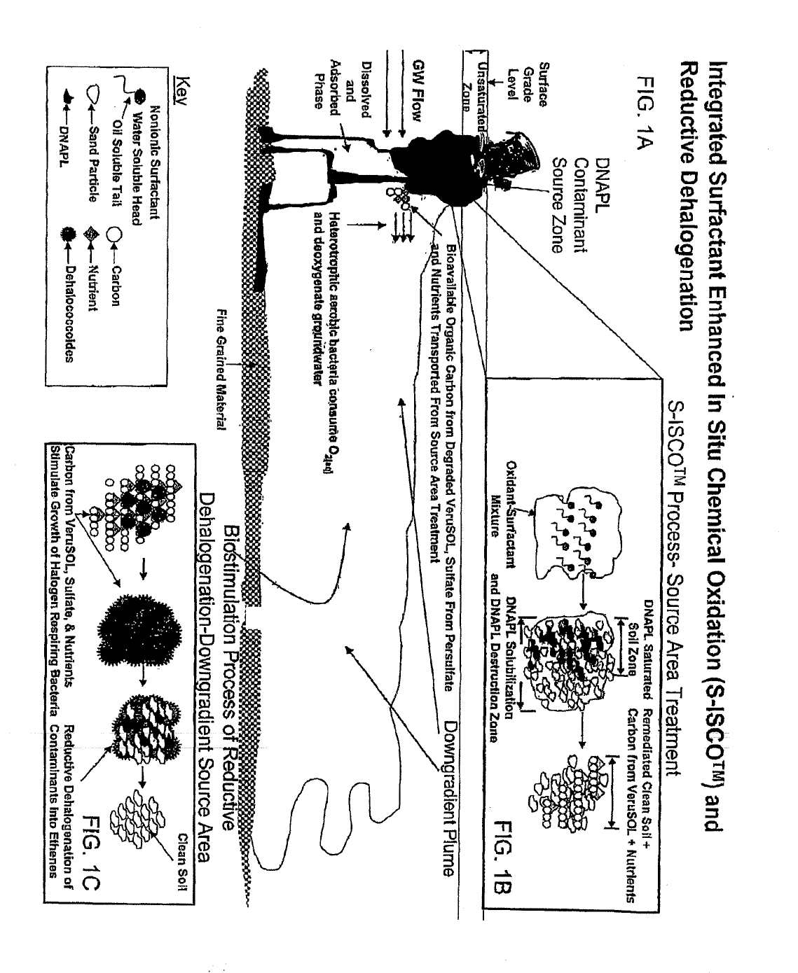

### DETAILED DESCRIPTION

Embodiments of the invention are discussed in detail below. In describing embodiments, specific terminology is employed for the sake of clarity. However, the invention is not intended to be limited to the specific terminology so selected. A person skilled in the relevant art will recognize that other equivalent parts can be employed and other methods developed without parting from the spirit and scope of the invention. All references cited herein are incorporated by reference as if each had been individually incorporated.

### S-ISCO™

Surfactant enhanced in-situ chemical oxidation (S-ISCO™) remediation can be used to remediate sites, for example, subsurfaces, in which soil in a subsurface is contaminated by chemicals of concern (COCs), such as non-aqueous phase liquids (NAPLs). Surfactant or surfactant-cosolvent mixtures can be injected to create an effective solubilized micelle or microemulsion, for example, a Windsor I system, with the COC present in the soil. This solubilized micelle or microemulsion can increase the apparent solubility of the COC, so that the solubilized micelle or microemulsed COC is able to enter into “aqueous phase reactions.” In the case of S-ISCO™ remediation, the COC can be oxidized using a primary oxidant such as permanganate, ozone, persulfate, activated persulfate, percarbonate, activated percarbonate, or hydrogen peroxide, or ultraviolet (uV) light or any combination of these oxidants with or without uV light. Several methods can be used to activate or catalyze peroxide and persulfate to form free radicals such as free or chelated transition metals and uV light. Persulfate can be additionally activated at both high and low pH, by heat or by peroxides, including calcium peroxides. Persulfate and ozone can be used in a dual oxidant mode with hydrogen peroxide.

Although remediation systems that rely on Winsor Type I solubilized micelle or microemulsification can be less efficient than those that rely on Winsor Type III microemulsions and mobilization, the desorption and solubilization of contaminants using Winsor Type I microemulsions are controllable such that the risk of off-site mobilization of NAPL chemicals of concern (COCs) is minimal and that complete recovery of injected chemicals, mobilized COC phases, and solubilized COC or sorbed chemicals using extraction wells is not required. Under solubilizing conditions, the NAPL removal rate can be dependent on the increase in solubility of the NAPL in the surfactant mixture. Under desorbing conditions, the sorbed COC species removal rate can be dependent on the rate of desorption of the COC into the surfactant or surfactant-cosolvent mixture.

The S-ISCO™ process includes a method and process of increasing the solubility of contaminants, such as normally low solubility nonaqueous phase liquids (NAPLs), sorbed contaminants, or other chemicals in soils in surface and ground water. Simultaneously or subsequently, the contaminating chemicals are oxidized using a chemical oxidant without the need of extraction wells for the purpose of recovering the injected cosolvents and/or surfactants with NAPL compounds. Examples of contaminants are dense nonaqueous phase liquids (DNAPLs), light nonaqueous phase liquids (LNAPLs), nonaqueous phase liquids (NAPLs), sorbed contaminants, polycyclic aromatic hydrocarbons (PAHs), chlorinated solvents, pesticides, herbicides, polychlorinated biphenyls, and various organic chemicals, such as petroleum hydrocarbons and their products. Contaminants can be associated with, for example, manufactured gas plant residuals, creosote wood treating liquids, petroleum residuals, pesticide, or polychlorinated biphenyl (PCB) residuals and other waste products or byproducts of industrial processes and commercial activities. Contaminants may be in the liquid phase, for example, NAPLs, sorbed to the soil matrix, or in the solid phase, for example, certain pesticides.

A subsurface can include any and all materials below the surface of the ground, for example, groundwater, soils, rock, man-made structures, naturally occurring or man-made contaminants, waste materials, or products. Knowledge of the distribution of hydraulic conductivity in the soil and other physical hydrogeological subsurface properties, such as hydraulic gradient, saturated thickness, soil heterogeneity, and soil type is desirable to determine, for example, the relative contribution of downward vertical density driven flow to normal advection in the subsurface.

The composition of a surfactant and/or cosolvent liquid amendment for injection into a subsurface can include a natural surfactant or a surfactant derived from a natural product, such as a plant oil or plant extract. Mixtures of these natural surfactants or surfactants derived from natural products can be chosen to best emulsify the subsurface contaminant, e.g., NAPL, LNAPL, or DNAPL, such that a mobile phase emulsion is formed with greatly differing properties from the source contaminant. The choice of surfactants and/or cosolvents can be based on the testing of the source contaminant. For example, a surfactant and/or cosolvent mixture can be selected to produce a low interfacial tension that enables the formation of either Winsor Type I, Winsor Type II, or Winsor Type III systems. A preferred formation of microemulsions is to form Winsor Type III microemulsions or Winsor Type I microemulsions. Frequently the preferred natural solvent such as those derived from plants are generally biodegradable, including terpenes. Terpenes are natural products extracted from conifer and citrus plants, as well as many other essential oil producing species. The combination of cosolvent and surfactants enhances the formation of microemulsions from a contaminant, e.g., a NAPL, LNAPL, or DNAPL. The specific choice of natural cosolvents and the ratio of cosolvent to surfactant can be based on laboratory tests conducted on the contaminant to be emulsified. All of the above natural surfactants, surfactants derived from natural oils and natural cosolvents can be combined into formulations to form non- or low-toxicity macroemulsions and microemulsions, with contaminants, enhancing their recovery, and, thus, elimination from a contaminated subsurface. Once emulsified, the contaminant-surfactant-cosolvent system is formed, so that the contaminant is amenable to become mobile in the subsurface.

For example, a method for reducing the concentration of a contaminant at a site in a subsurface can include the following. The contaminant can, for example, include a non-aqueous phase liquid (NAPL), a dense non-aqueous phase liquid (DNAPL), and/or a light non-aqueous phase liquid (LNAPL). An extraction well can be provided in the subsurface; for example, the extraction well can remove bulk quantities of contaminant. An injection fluid can be injected at an injection locus into the subsurface. The injection fluid can include hydrogen peroxide and/or another oxidant, or another gas phase generating oxidant or pressure dissolved gas in a liquid. The hydrogen peroxide and/or the other oxidant or dissolved gas can be allowed to decompose to liberate oxygen or dissolved gas in the subsurface. The other oxidant can be, for example, ozone, a persulfate, sodium persulfate, or a percarbonate. The injection fluid can include a liquid, e.g., water, and a dissolved gas, e.g., oxygen and/or carbon dioxide, and the dissolved gas can effervesce as a liberated gas upon a decrease of pressure on the injection fluid in the subsurface. The injection fluid can include a compressed gas and/or a supercritical fluid under a pressure greater than atmospheric. An injected gas can include, for example, oxygen, carbon dioxide, nitrogen, air, an inert gas, helium, argon, another gas, or combinations of these. The injection fluid can include a surfactant and/or a cosolvent, for example, the injection fluid can include VeruSOL. The injection fluid can include an alkali carbonate or bicarbonate, such as sodium bicarbonate. The injection fluid can include an activator, for example, a metal activator, a chelated metal activator, a chelated iron activator, Fe-NTA, Fe(II)-EDTA, Fe(III)-EDTA, Fe(II)-citric acid, or Fe(III)-citric acid. The injection fluid can include an antioxidant. The oxygen and/or the gas produced from reaction of the oxygen, hydrogen peroxide, and/or other oxidant with the contaminant, e.g., carbon dioxide, can be allowed to impose pressure to force the contaminant to flow through the subsurface toward the extraction well. The contaminant can be removed from the extraction well to a surface above the subsurface. The contaminant can then be stored, for example, in a storage tank, or can be disposed of, for example, in a waste destruction facility. A wide range of configurations can be used to implement facilitated remediation by extraction aided by gas pressure in conjunction with ISCO or S-ISCO. The selection of a configuration for remediation of a site can be guided by considerations of, for example, the nature of the contaminant, hydrogeology of the site, economics of procedures such as well drilling and waste disposal, and costs of chemicals such as oxidants, cosolvents, and surfactants.

In implementing S-ISCO, the surfactant or surfactant-cosolvent mixture can be introduced sequentially or simultaneously (together) with the oxidant into a subsurface. For example, the surfactant or surfactant-cosolvent mixture can first be introduced, then the oxidant and/or other injectants can be introduced. Alternatively, the oxidant can first be introduced, then the surfactant or surfactant-cosolvent mixture can be introduced. Alternatively, the oxidant and the surfactant or surfactant-cosolvent mixture can be introduced simultaneously. Simultaneously can mean that the oxidant and the surfactant and/or cosolvent are introduced within 6 months of each other, within 2 months of each other, within 1 month of each other, within 1 week of each other, within 1 day of each other, within one hour of each other, or together, for example, as a mixture of oxidant with surfactant and/or cosolvent. In each case, the oxidant is present in sufficient amounts at the right time, together with the surfactant, to oxidize contaminants as they are solubilized or mobilized by surfactant or cosolvent-surfactant mixture.

For example, shallow contamination near the water table can be effectively targeted by using persulfate concentrations in the, say, 10 g/L (grams per liter) to 15 g/L range and moderately high injection flowrates, e.g., up to 30 gpm (gallons per minute) per injection location, dependent on the geometry of the injection trench or wells. For intermediate depth locations, persulfate concentrations up to, say, 25 g/L can be used with, e.g., up to 20 gpm per injection, dependent on the geometry of the injection trench or wells. For deeper DNAPL contamination, persulfate concentrations up to 100 g/L can be used dependent on the nature of the DNAPL distributions and concentrations. Injection flowrates for deep DNAPL applications can be up to, say, 20 gpm per well, if injected above the lower permeability layers and up to, say, 10 gpm per well, if injected in the lower permeability unit. For certain subsurfaces, it may be advantageous to inject substances such as oxidants, surfactants, and/or cosolvents under elevated pressure. Unlike permanganate, persulfate forms no significant solid phase precipitates.

A goal in the remediation of sites containing large quantities of contaminants, such as LNAPLs and DNAPLs, is to obtain the benefits of ISCO (in-situ chemical oxidation) or S-ISCO (surfactant enhanced in-situ chemical oxidation) in destroying the contaminants without mobilizing them off site, while reducing the quantity and thus the cost of the oxidant injected.

In an embodiment, a user creates a localized zone in the subsurface for the extraction of large quantities of contaminants, such as LNAPLs or DNAPLs (extraction zone), while having chemical oxidation of the contaminants take place in the subsurface beyond the extraction zone. The contaminants extracted may either be in a phase-separated state or in a solubilized or emulsified state. By creating a zone of chemical oxidation of the contaminants beyond the localized extraction zone, the risks associated with incomplete extraction of contaminants, such as LNAPLs or DNAPLs, inherent in traditional SEAR (surfactant-enhanced aquifer remediation) applications are minimized or eliminated. That is, in a process according the invention, a zone of chemical oxidation (oxidation zone), a zone of biodegradation by aerobic microorganisms, and/or a zone of biodegradation by anaerobic microorganisms surrounding the extraction zone serves to destroy any contaminant that migrates out of the extraction zone, and thus prevents the spread of contaminant. Thus, the simultaneous use of the S-ISCO (surfactant enhanced in-situ chemical oxidation) process with extraction of the solubilized or emulsified LNAPLs and/or DNAPLs minimizes the risk from migration of NAPLs.

At the same time, by employing liquid extraction using single and/or dual phase pumping systems, for example, of the types that are commonly known in the art, the amount of oxidant chemical required may be less than that when ISCO (in-situ chemical oxidation) or S-ISCO (surfactant enhanced in-situ chemical oxidation) is used alone. At sites with large quantities of LNAPLs and/or DNAPLs, the cost of liquid extraction of contaminants, such as LNAPLs and/or DNAPLs, coupled with ISCO or S-ISCO may be less than using ISCO or S-ISCO alone. That is, the cost of extraction and subsequent on site treatment or off-site disposal of the contaminants may be offset by the savings represented by the decrease in the quantity of oxidant and/or other chemicals required. Thus, sites containing large quantities of contaminants, such as LNAPLs or DNAPLs, can be cost-effectively treated.

Additional description of the S-ISCO™ process can be found in the international application PCT/US2007/007517, published as WO2007/126779, which is hereby incorporated by reference.

### Surfactants and Cosolvents

Surfactant or surfactant-cosolvent mixtures to solubilize NAPL components and desorb contaminants of concern (COCs) from site soils or from NAPL in water mixtures can be screened for use in a combined surfactant-oxidant treatment. For example, blends of biodegradable citrus-based solvents (for example, d-limonene) and degradable surfactants derived from natural oils and products can be used.

For example, a composition of surfactant and cosolvent can include at least one citrus terpene and at least one surfactant. A citrus terpene may be, for example, CAS No. 94266-47-4, citrus peels extract (citrus spp.), citrus extract, Curacao peel extract (Citrus aurantium L.), EINECS No. 304-454-3, FEMA No. 2318, or FEMA No. 2344. A surfactant may be a nonionic surfactant. For example, a surfactant may be an ethoxylated castor oil, an ethoxylated coconut fatty acid, or an amidified, ethoxylated coconut fatty acid. An ethoxylated castor oil can include, for example, a polyoxyethylene (20) castor oil, CAS No. 61791-12-6, PEG (polyethylene glycol)-10 castor oil, PEG-20 castor oil, PEG-3 castor oil, PEG-40 castor oil, PEG-50 castor oil, PEG-60 castor oil, POE (polyoxyethylene) (10) castor oil, POE(20) castor oil; POE (20) castor oil (ether, ester); POE(3) castor oil, POE(40) castor oil, POE(50) castor oil, POE(60) castor oil, or polyoxyethylene (20) castor oil (ether, ester). An ethoxylated coconut fatty acid can include, for example, CAS No. 39287-84-8, CAS No. 61791-29-5, CAS No. 68921-12-0, CAS No. 8051-46-5, CAS No. 8051-92-1, ethoxylated coconut fatty acid, polyethylene glycol ester of coconut fatty acid, ethoxylated coconut oil acid, polyethylene glycol monoester of coconut oil fatty acid, ethoxylated coco fatty acid, PEG-15 cocoate, PEG-5 cocoate, PEG-8 cocoate, polyethylene glycol (15) monococoate, polyethylene glycol (5) monococoate, polyethylene glycol 400 monococoate, polyethylene glycol monococonut ester, monococonate polyethylene glycol, monococonut oil fatty acid ester of polyethylene glycol, polyoxyethylene (15) monococoate, polyoxyethylene (5) monococoate, or polyoxyethylene (8) monococoate. An amidified, ethoxylated coconut fatty acid can include, for example, CAS No. 61791-08-0, ethoxylated reaction products of coco fatty acids with ethanolamine, PEG-11 cocamide, PEG-20 cocamide, PEG-3 cocamide, PEG-5 cocamide, PEG-6 cocamide, PEG-7 cocamide, polyethylene glycol (11) coconut amide, polyethylene glycol (3) coconut amide, polyethylene glycol (5) coconut amide, polyethylene glycol (7) coconut amide, polyethylene glycol 1000 coconut amide, polyethylene glycol 300 coconut amide, polyoxyethylene (11) coconut amide, polyoxyethylene (20) coconut amide, polyoxyethylene (3) coconut amide, polyoxyethylene (5) coconut amide, polyoxyethylene (6) coconut amide, or polyoxyethylene (7) coconut amide.

Examples of surfactants and/or cosolvents that can be used include terpenes, citrus-derived terpenes, limonene, d-limonene, castor oil, coca oil, coconut oil, soy oil, tallow oil, cotton seed oil, and a naturally occurring plant oil. The surfactant and/or cosolvent can be a nonionic surfactant, such as ethoxylated soybean oil, ethoxylated castor oil, ethoxylated coconut fatty acid, and amidified, ethoxylated coconut fatty acid. The surfactant and/or cosolvent can be ALFOTERRA 123-8S, ALFOTERRA 145-8S, ALFOTERRA L167-7S, ETHOX HCO-5, ETHOX HCO-25, ETHOX CO-5, ETHOX CO-40, ETHOX ML-5, ETHAL LA-4, AG-6202, AG-6206, ETHOX CO-36, ETHOX CO-81, ETHOX CO-25, ETHOX TO-16, ETHSORBOX L-20, ETHOX MO-14, S-MAZ 80K, T-MAZ 60 K 60, TERGITOL L-64, DOWFAX 8390, ALFOTERRA L167-4S, ALFOTERRA L123-4S, and ALFOTERRA L145-4S.

Examples of surfactants derived from natural plant oils are ethoxylated coca oils, coconut oils, soybean oils, castor oils, corn oils and palm oils. Many of these natural plant oils are US FDA GRAS.

Compositions for use as surfactant and/or cosolvent liquid amendments for subsurface injection can include natural biodegradable surfactants and cosolvents. Natural biodegradable surfactants can include those that occur naturally, such as yucca extract, soapwood extract, and other natural plants that produce saponins, such as horse chestnuts (Aesculus), climbing ivy (Hedera), peas (Pisum), cowslip, (Primula), soapbark (Quillaja), soapwort (Saponaria), sugar beet (Beta) and balanites (Balanites aegyptiaca). Many surfactants derived from natural plant oils are known to exhibit excellent surfactant power, and are biodegradable and do not degrade into more toxic intermediary compounds.

Examples of cosolvents which preferentially partition into the NAPL phase include higher molecular weight miscible alcohols such as isopropyl and tert-butyl alcohol. Alcohols with a limited aqueous solubility such as butanol, pentanol, hexanol, and heptanol can be blended with the water miscible alcohols to improve the overall phase behavior. Given a sufficiently high initial cosolvent concentration in the aqueous phase (the flooding fluid), large amounts of cosolvent partition into the NAPL. As a result of this partitioning, the NAPL phase expands, and formerly discontinuous NAPL ganglia can become continuous, and hence mobile. This expanding NAPL phase behavior, along with large interfacial tension reductions, allows the NAPL phase to concentrate at the leading edge of the cosolvent slug, thereby increasing the mobility of the NAPL. Under certain conditions, a highly efficient piston-like displacement of the NAPL is possible. Because the cosolvent also has the effect of increasing the NAPL solubility in the aqueous phase, small fractions of the NAPL which are not mobilized by the above mechanism are dissolved by the cosolvent slug.

The surfactants/cosolvents Citrus Burst 1, Citrus Burst 2, Citrus Burst 3, and E-Z Mulse are manufactured by Florida Chemical.

### Combined S-ISCO™ and Biodegradation

The S-ISCO process can be performed in combination with a biodegradation (bioremediation) process. Combining S-ISCO and biodegradation processes can result in several advantageous synergies. For example, a surfactant, such as VeruSOL can gives give an oxidant access to contaminants, so that the oxidant oxidizes the contaminant. A surfactant, such as VeruSOL, can then provide a source of carbon as well as nitrogen and phosphorous nutrients to stimulate proliferation of native microorganisms. For example, persulfate can provide a source of electron acceptor to “sulfideogenic bacteria” that then can further break down contaminants. Other sources of carbon for microorganisms include organic compounds partially degraded by injected (e.g. persulfate) oxidant; the compounds may be more accessible to the microorganisms in a partially degraded state. Other sources of carbon for microorganisms include undegraded organic contaminant compounds and naturally present subsurface organic compounds.

The S-ISCO part of a combined process can allow for faster elimination of contaminants than bioremediation alone. The bioremediation part of a combined process can supplement S-ISCO by further degrading contaminant partially oxidized by S-ISCO, and can complement S-ISCO, for example by degrading contaminants that may be resistant to chemical oxidation. The bioremediation part of a combined process can also reduces the amount required of injected remediation chemicals, e.g., oxidant and surfactant, which can reduce the cost of remediation. Furthermore, the bioremediation part of a combined process can eliminate residual surfactant and residual oxidant remaining after destruction of the contaminant. This can prevent the residual surfactant and residual oxidant from becoming contaminants themselves, and can allow for the use of excess surfactant and oxidant at the beginning of a remediation process so as to achieve faster remediation.

Some observations in a S-ISCO pilot test in the field of a remediation process indicate that contaminant levels and persulfate levels continue to decrease after the injection of persulfate oxidant ceases, and may indicate that native microorganisms biodegrade contaminant and may have been stimulated by the injected substances.

Surfactant enhanced in-situ chemical oxidation (S-ISCO™) remediation of hydrocarbon contaminants can yield products other than those associated with complete oxidation. For example, S-ISCO™ treatment of a high-molecular weight hydrocarbon can yield partially oxidized hydrocarbons of intermediate molecular weight, rather than only carbon dioxide and water.

In an embodiment, partially oxidized hydrocarbons can be allowed to remain in the soil, where they are further degraded to lower molecular weight species by microorganisms in the soil. For example, an oxidant such as sodium persulfate can be injected into the soil where a contaminant is present to induce primary or chemical oxidation. The oxidant may completely or only partially oxidize the contaminant. The partially oxidized contaminant can, for example, diffuse to a different region, be displaced by a convective flow, for example, the flow of groundwater, or remain where it is. For example, the subsurface can have a downgradient direction and can include groundwater. The groundwater can flow in the downgradient direction, and transport the partially oxidized contaminant with it. For example, downgradient of the point or locus where the oxidant is introduced can refer to at least about 150 ft, 200 ft, 250 ft, or 275 feet downgradient of the oxidant injection locus. Alternatively, the injection of material at a point, for example, surfactant, oxidant, and water at the source of contamination, can flow outward from the point, and transport partially oxidized contaminant with it.

Microorganisms present in the soil, such as aerobic or anaerobic microbes, can further oxidize the contaminant or reductively break down the contaminant.

Combining biodegradation with the injection of a primary oxidant, such as sodium persulfate, can provide a number of benefits. The primary oxidant can induce the rapid chemical oxidation of the contaminant. Even if the contaminant is not fully oxidized, the partially oxidized products can be less harmful than the initial contaminant.

By contrast, a biodegradation approach alone may proceed rather slowly. Biodegradation reactions can take place in the dissolved phase, and the low solubility of organic contaminants in the dissolved phase and the slow rate of their transfer to the dissolved phase can limit the overall rate of biodegradation. Furthermore, the rate at which microorganisms metabolize contaminants can be slow, so that even if a surfactant is used to increase the rate of mass transfer to the dissolved phase, the rate of biodegradation can still be slow. Because of the slow rate of biodegradation, the dissolved contaminants can be mobilized away from the initial site of contamination. In so spreading, the dissolved contaminants can cause harm before they are biodegraded. Thus, a biodegradation approach alone may not be optimal for treating the source zone of a contaminated site, which has a high concentration of contaminants that cannot be sufficiently rapidly destroyed by biodegradation alone. The slow progress of anaerobic degradation can render remediation unacceptable from a practical or business standpoint.

Furthermore, for an exclusively reductive biodegradation process, a large amount of material required to serve as food for aerobic microorganisms may need to be provided. For anaerobic microorganisms, a material that can react with oxygen (which can be food for aerobic microorganisms that causes the aerobic microorganisms to consume oxygen) may need to be added in order to consume available oxygen and establish a reductive environment.

However, biodegradation can be a valuable adjuvant to the S-ISCO™ process. For example, an oxidative environment may be present away from the site of primary injection where the majority of chemical oxidation proceeds. Even if strict chemical oxidation does not take place at a sufficient rate in this removed oxidative environment, certain aerobic microorganisms in the soil can make use of the available oxygen to degrade remaining unoxidized contaminant or partially oxidized contaminant. The use of such aerobic microorganisms may result in a more complete destruction of the contaminant, may allow for the amount of primary oxidant injected to be reduced, thus reducing cost, or may allow for treatment by biodegradation of contaminant to continue far from the site of injection of the primary oxidant, for example, when groundwater carries contaminant downgradient in a plume.

Certain contaminants, for example, halogenated hydrocarbons, may exhibit some resistance to degradation by chemical oxidation. In such a case, chemical oxidation induced by injection of a primary oxidant, such as sodium persulfate, can be complemented by reduction of the remaining contaminants or products by anaerobic microorganisms in a region with a reducing environment removed from the region where chemical oxidation occurs.

In a combined S-ISCO™—biodegradation approach, chemical addition can be limited to the contaminant source zone. The organic carbon dissolved by VeruSOL and degraded COCs and nutrients from VeruSOL can then migrate downgradient in the plume. S-ISCO™ can solubilize NAPLs rapidly and simultaneously and rapidly oxidize the solubilized compounds. Any unoxidized or partially oxidized compounds remaining from S-ISCO™ can consume available oxygen to create reducing zones in which anaerobic processes like reductive dechlorination can take place.

For example, the destruction of contaminants in soil in the subsurface can proceed as follows. The primary oxidant injected can partially or fully oxidize contaminants susceptible to oxidation, such as aromatic hydrocarbons. Contaminants resistant to oxidation, such as certain halogenated organics, may be only partially oxidized or not oxidized at all. At some distance from the primary oxidant injection site, the concentration of oxygen can decrease, so that although an oxidizing environment is still present, the rate of chemical oxidation is low. In this region, aerobic microorganisms can continue to degrade contaminant. Still farther from the primary oxidant injection site, the oxidation potential can decrease, for example, can decrease to less than zero, so that a reducing environment is present. In the reducing environment, anaerobic microorganisms can degrade contaminants, for example, contaminants that are resistant to oxidation, such as halogenated organics.

S-ISCO™ generates products of partial oxidation that are easy to biodegrade compounds (and can serve as a food source for microorganisms) from both contaminants and from naturally biodegradable cosolvents and surfactants used for S-ISCO™. As the natural cosolvents and surfactants used undergo oxidation, they become more easily naturally biodegraded. The production of easy to biodegrade compounds from both the organic liquids and the cosolvent/surfactants may require more electron acceptors (i.e., oxygen in the case of aerobic biodegradation and in the case of anaerobic biodegradation alternative electron acceptors).

Nutrients, for example, an inorganic or an organic substance, for example, a micronutrient or a macronutrient, can be injected into the subsurface to stimulate the growth of microorganisms, for example, aerobic or anaerobic microorganisms. Such a nutrient can be a mineral, for example, potassium persulfate or ammonium persulfate. Thus, the primary oxidant can also serve as a nutrient. For example, the surfactants injected as part of the S-ISCO process can serve as a source of food and/or nutrients for microorganisms. For example, the plant-derived surfactants present in VeruSOL can include carbon, nitrogen and phosphorous, which can be useful as food and nutrients to microorganisms. For example, oxidants injected can serve as a source of food and/or nutrients for microorganisms. For example, the oxidants potassium persulfate and/or ammonium persulfate can serve as nutrient sources of potassium and nitrogen. Thus, in designing a remediation process, the selection of compounds used for S-ISCO, such as surfactants and oxidants, can be influenced by the synergistic role they can play in promoting the growth and metabolism of microorganisms that biodegrade contaminants and/or partially oxidized products of contaminants.

Furthermore, in designing a remediation process, certain compounds can be introduced to the site to be remediated, for example, a subsurface, primarily for their role in promoting the growth and metabolism of microorganisms. The nutrient can include an organic or inorganic (e.g., mineral) compound or other substance that promotes the growth of microorganisms. For example, macronutrients and/or micronutrients can be injected. For example, at some distance from a locus where a primary oxidant is injected for S-ISCO, a reductant can be injected to induce a reductive environment to promote the growth and metabolism of anaerobic microorganisms. Alternatively, at some distance from a locus where a primary oxidant is injected for S-ISCO, an oxidant can be injected to induce an oxidative environment to promote the growth and metabolism of aerobic microorganisms.

S-ISCO™ can be used to create source zone treatment. At the same time, S-ISCO™ can generate easily biodegradable organic chemicals from contaminants and solubilize organic NAPLs, to promote the biodegradation treatment of soil and groundwater plumes located downgradient of a source zone.

The coupling of S-ISCO™ with bioremediation for remediation overcomes several major limitations of bioremediation alone. The S-ISCO™ and bioremediation coupled approach overcomes NAPL solubility and desorption limitations that restrict mass transfer to the aqueous phase. S-ISCO™ can rapidly break down environmental contaminants, and lessen the amount of total contaminant that the slower biodegradation processes need to break down. Many environmental contaminants are difficult to biodegrade, but their oxidation products can be readily biodegraded; thus, S-ISCO™ can produce oxidation products that are then biodegraded.

The oxidation rates of compounds associated with source area organic liquids, NAPLs, and sorbed COCs using S-ISCO™ are much faster than those achievable using biodegradation alone in the subsurface. Downgradient from source area S-ISCO™ treatment, it may be cost effective to use natural or stimulated biodegradation for contaminated plume management and treatment.

Surfactants or mixtures of cosolvents and surfactants together with chemical oxidants can be used to solubilize and oxidize organic liquids, NAPLs, and/or sorbed organic chemicals in contaminant source zones. These organic liquids, NAPLs, and/or sorbed organic chemicals and/or their oxidation products can then subsequently be biologically or enzymatically degraded, either aerobically or anaerobically. Naturally biodegradable surfactants or cosolvent/surfactants can be used, so that these can be aerobically or anaerobically biodegraded along with the organic liquids, NAPLs, and/or sorbed organic chemicals and/or their oxidation products.

Some contaminants when partially oxidized can be more susceptible to degradation by aerobic microorganisms, whereas other contaminants when partially oxidized can be more susceptible to degradation by anaerobic microorganisms. For example, non-halogenated aromatic hydrocarbons and partially oxidized non-halogenated aromatic hydrocarbons can be degraded by aerobic microorganisms. On the other hand, halogenated organics and partially oxidized halogenated organics can be degraded by anaerobic microorganisms. Thus, an approach to performing in-situ remediation of a contaminant can be developed based on the type of the contaminant present in a subsurface.

In an embodiment, a microorganism can be introduced into the subsurface, for example, to “seed” the subsurface and thereby promote biodegradation. For example, an aerobic microorganism and/or an aerobic microorganism can be introduced into the subsurface.

Microorganisms such as Burkholderia, Burkholderia xenovorans, Rhodococcus, Aromatoleum aromaticum, Geobacter metallireducens, Dechloromonas aromatica, Dehalococcoides, Dehalococcoides ethenogenes, Desulfitobacterium hafniense, Pseudomonas, Pseudomonas alcaligenes, Pseudomonas mendocina, Pseudomonas resinovorans, Pseudomonas veronii, Pseudomonas putida, Pseudomonas stutzeri, Deinococcus radiodurans are capable of biodegrading hydrocarbons, such as aromatic hydrocarbons and halogenated hydrocarbons.

For example, S-ISCO and aerobic and/or anaerobic biodegradation can be used to reduce the amount or concentration of a contaminant at a site, e.g., in a subsurface, to a predetermined level. For example, a predetermined level of a contaminant can be a maximum amount of a contaminant at a site permitted by law or can be a maximum concentration of a contaminant permitted by law.

### Oxidative Zones: Oxidants, Activators, and Antioxidants

Oxygen sources for chemical oxidation of contaminants and/or for the stimulation of aerobic biodegradation of contaminants can come from injection of oxygen from air or pure oxygen, ozone, solid oxides, and peroxides or other means of introducing oxygen into the subsurface or above ground. For example, a persulfate, sodium persulfate, a percarbonate, a permanganate, potassium permanganate, and hydrogen peroxide can be introduced into the subsurface or above ground to provide a source of oxygen.

If a targeted contaminant can be degraded by aerobic microorganisms, the growth of aerobic microorganisms can be encouraged by establishing an oxidative zone or environment.

Aerobic metabolism by aerobic microorganisms can be represented by the reaction O₂+4e⁻+4H⁺→2H₂O. Aerobic metabolism can take place, for example, when the environment has a redox potential, Eh, of from about 600 to about 400 mV.

For example, in an oxidative environment, aerobic microbes can further oxidize partially oxidized contaminant that is a product of treatment of contaminant with an oxidant. Such aerobic degradation of partially oxidized contaminant can be stimulated by, for example, introducing an oxidant at a different time or location or of a different type than associated with the oxidant used to initially oxidize the contaminant. For example, at a treatment site where contaminant is moved by groundwater flow, a secondary oxidant, such as hydrogen peroxide can be injected at a second injection point downgradient of the point where the primary oxidant used to initially oxidize the compound, e.g., sodium persulfate, was added. The injection of hydrogen peroxide can induce or maintain an oxidative environment in the subsurface, and stimulate the growth and activity of aerobic microorganisms that break down contaminant. A secondary oxidant can be the same as or different from a primary oxidant.

An activator can be, for example, a chemical molecule or compound, or another external agent or condition, such as heat, temperature, or pH, that increases the rate of or hastens a chemical reaction. The activator may or may not be transformed during the chemical reaction that it hastens. Examples of activators which are chemical compounds include a metal, a transition metal, a chelated metal, a complexed metal, a metallorganic complex, and hydrogen peroxide. Examples of activators which are other external agents or conditions include heat, temperature, and high pH. Preferred activators include Fe(II), Fe(III), Fe(II)-EDTA, Fe(III)-EDTA, Fe(II)-citric acid, Fe(III)-citric acid, hydrogen peroxide, high pH, and heat. Zero valent metals, such as zero valent iron, can serve as activators of oxidants, for example, hydrogen peroxide.

Non-thermal ISCO using persulfate can be activated by ferrous ions or by preferentially chelated metals. Chelated iron has been demonstrated to prolong the activation of persulfate enabling activation to take place at substantial distances from injection wells.

Several practical sources of Fe(II) or Fe(III) can be considered for activation of persulfate. Iron present in the soil minerals that can be leached by injection of a free-chelate (a chelate not complexed with iron, but usually Na⁺ and H⁺) can be a source. Injection of soluble iron as part of a chelate complex, such as Fe(II)-EDTA, Fe(II)-NTA or Fe(II)-Citric Acid (other Fe-chelates are available) can be a source. Indigenous dissolved iron resulting from reducing conditions present in the subsurface (common at many MGP sites) can be a source. For the Pilot Test, discussed as an example below, Fe(II)-EDTA was used.

An example of an oxidant is persulfate, e.g., sodium persulfate, of an activator is Fe(II)-EDTA, of a surfactant is Alfoterra 53, and of a cosolvent-surfactant mixture is a mixture of d-limonene and biodegradable surfactants, for example, Citrus Burst 3. Citrus Burst 3 includes a surfactant blend of ethoxylated monoethanolamides of fatty acids of coconut oil and polyoxyethylene castor oil and d-limonene.

An embodiment of the invention is the simultaneous or sequential use of the oxidant persulfate, and an activator to raise the pH of the groundwater to above 10.5 by the addition of CaO, Ca(OH)₂, NaOH, or KOH, an example of a cosolvent-surfactant is Citrus Burst 3.

An aspect of the control that can be achieved by use of an embodiment of the invention for site remediation is direction of antioxidant to a target region of contaminant. The density of the injected solution can be modified, so that the oxidant reaches and remains at the level in the subsurface of the target region of contaminant. Additional factors such as subsurface porosity and groundwater flow can be considered to locate wells for injecting solution containing oxidant, so that oxidant flows to the target region of contaminant.

In an embodiment, the consumption of oxidant can be controlled by including an antioxidant in the injected solution. For example, an antioxidant can be used to delay the reaction of an oxidant. Such control may prove important when, for example, the injected oxidant must flow through a region of organic matter which is not a contaminant and with which the oxidant should not react. Avoiding oxidizing this non-contaminant organic matter may be important to maximize the efficiency of use of the oxidant to eliminate the contaminant. That is, if the oxidant does not react with non-contaminant organic matter, then more oxidant remains for reaction with the contaminant. Furthermore, avoiding oxidizing non-contaminant organic matter may be important in its own right. For example, topsoil or compost may be desirable organic matter in or on soil that should be retained. The anti-oxidants used may be natural compounds or derivatives of natural compounds. By using such natural antioxidants, their isomers, and/or their derivatives, the impact on the environment by introduction of antioxidant chemicals is expected to be minimized. For example, natural processes in the environment may degrade and eliminate natural antioxidants, so that they do not then burden the environment. The use of natural antioxidants is consistent with the approach of using biodegradable surfactants, cosolvents, and solvents. An example of a natural antioxidant is a flavonoid. Examples of flavonoids are quercetin, glabridin, red clover, Isoflavin Beta (a mixture of isoflavones available from Campinas of Sao Paulo, Brazil). Other examples of natural antioxidants that can be used as antioxidants in the present method of soil remediation include beta carotene, ascorbic acid (vitamin C), and tocopherol (vitamin E) and their isomers and derivatives. Non-naturally occurring antioxidants, such as beta hydroxy toluene (BHT) and beta hydroxy anisole (BHA) can also be used as antioxidants in the present method of soil remediation.

For compounds that aerobically degrade, such as petroleum hydrocarbons, oxygen, for example, in the form of a peroxide, can be added with persulfate and Verusol to dissolve and chemically oxidatively degrade petroleum hydrocarbons in the source area. The oxygen contributed by the peroxides can then induce the biodegradation by aerobic microorganisms of petroleum hydrocarbons in the downgradient dissolved plume. Thermodynamically, aerobic degradation reaction rates are much faster than anaerobic reaction rates.

Compounds of incomplete oxidation of petroleum or coal-derived materials from the S-ISCO™ process and naturally aerobically degradable cosolvent/surfactant chemicals used in the S-ISCO™ process, as well as their oxidation products, can be biodegraded. Air, oxygen, solid peroxide compounds, and liquid peroxide compounds can be added to enhance aerobic biodegradation. However, traditional approaches are limited in that they only treat the dissolved phase and do not treat the NAPL or organic liquid phase. Certain contaminants can be difficult to biodegrade and require complex biological systems difficult to sustain in the subsurface. For example, certain PAH compounds require lignin degrading white rot fungi to partially biologically degrade PAH compounds to more readily degradable metabolites that are easier to biodegrade (Zheng and Obbard (JEQ, 31:2002). Sorbed PAHs in soils are not bioavailable and, therefore, not available to be biodegraded in the aqueous phase (Guha and Jaffee (1996)). Yeom and Ghosh (1998) concluded than mass transfer of PAHs from the sorbed phase into the aqueous phase limited biodegradation rates of PAHs and not biodegradation rate kinetics.

However, in the S-ISCO™ approach, the aerobically biodegradable cosolvents and surfactants increase the rate of solubilization of organics and NAPLs, and thereby greatly increase the rate and potential efficiency of aerobic degradation of NAPLs. For example, the S-ISCO™ process overcomes PAH mass transfer limitations into the aqueous phase and simultaneously chemically oxidizes the PAH compounds into either carbon dioxide and water or more easily aerobically biodegradable compounds. Coupling VeruTEK's S-ISCO™ process with oxygenation (by any of the means listed above) for the treatment of NAPL source areas and downgradient dissolved plumes is a significant enhancement over S-ISCO™ alone or oxygenation for biodegradation alone. Depending on the amount of NAPL compounds present and the ease with which the organic compounds are biodegraded, either VeruSOL alone or with plume oxygen addition can be used.

It can be important that the oxidant not react with the surfactant so fast that the surfactant is consumed before the surfactant can solubilize the contaminant. On the other hand, the surfactant should not reside in the subsurface indefinitely, to avoid being a contaminant itself. This degradation can be caused by living organisms, such as bacterial, through a biodegradation process. On the other hand, the surfactant can be selected to slowly react with the oxidant, so that the oxidant survives sufficiently long to solubilize the contaminant for the purpose of enhancing its oxidation, but once the contaminant has been oxidized, the surfactant itself is oxidized by the remaining oxidant.

At the oxidation zone, it may be acceptable for the oxidant to rapidly degrade the surfactant. If the surfactant is degraded, oxidation of the contaminant may be slowed, because only a small amount of contaminant is in the aqueous phase. However, at the same time, the contaminant may be effectively immobilized. This immobilization can prevent the contaminant from passing through the oxidation zone. Thus, even if the oxidant rapidly degrades the surfactant, the objective of preventing the contaminant from spreading beyond the oxidation zone may still be achieved.

For example, aerobic microorganisms can metabolize and consume excess oxidant, surfactant, cosolvent or another substance introduced that migrates away from the locus where the primary oxidant is injected. Thus, following a remediation procedure or outside of a region where remediation is to take place, aerobic microorganisms can reduce the amount or concentration of excess oxidant, surfactant, cosolvent or another substance introduced during the remediation process to a predetermined level, to ensure that such an introduced substance is not itself viewed as a contaminant. For example, a predetermined level of an introduced substance can be a maximum amount of an introduced substance at a site permitted by law or can be a maximum concentration of an introduced substance permitted by law.

### Reductive Zones

If a targeted contaminant contains a component degraded by anaerobic microorganisms, a two region (or two zone) approach can be developed. Near where the primary oxidant, for example, sodium persulfate, is injected, the primary oxidant will induce an oxidizing environment in the subsurface, and the primary oxidant will oxidize or partially oxidize portions of this contaminant. In this oxidizing environment, aerobic microorganisms can grow and break down components of the contaminant susceptible to degradation by aerobic microorganisms, such as non-halogenated aromatic hydrocarbons. The primary oxidant, along with any oxygen naturally present in the subsurface can be fully consumed in a reducing zone away from where the primary oxidant is injected. If the oxygen present is not fully consumed to form a reducing zone, a reducing zone can be induced by injecting a chemical that consumes oxygen at a point away from where the primary oxidant is injected. In this reducing zone, anaerobic microorganisms can grow and break down components of the contaminant susceptible to degradation by anaerobic microorganisms, such as halogenated organics.

Thus, when anaerobic degradation pathways to further destroy toxic organic chemical are preferred, the production of excess aerobically degradable compounds can lead to depletion of dissolved oxygen in the subsurface and create a low redox potential or reducing environment favorable for anaerobic metabolism or enzymatic reduction of organic and inorganic environmental contaminants. Such low redox environments resulting from the use of S-ISCO™ may result in inorganic reduction pathways associated with common inorganic redox couples, that facilitate organic and inorganic chemical reduction.

Anaerobic metabolism by anaerobic microorganisms can have several different forms. Several of these anaerobic metabolic processes, along with exemplary overall reactions and the redox potential, Eₕ, at which the processes can take place are represented in the table below.

| | | |
|---|---|---|
| denitrification | 2NO₃⁻ + 10e⁻ + 12H⁺ → N₂ + 6H₂O | 500~200 |
| manganese IV | MnO₂ + 2e⁻ + 4H⁺ → Mn²⁺ + 2H₂O | 400~200 |
| reduction | | |
| iron III reduction | Fe(OH)₃ + e⁻ + 3H⁺ → Fe²⁺ + 3H₂O | 300~100 |
| sulfate reduction | SO₄²⁻ + 8e⁻ + 10 H⁺ → H₂S + 4H₂O | 0~−150 |
| fermentation | 2CH₂O → CO₂ + CH₄ | −150~−220 |

For example, manganese-reducing bacteria can use reduction of manganese oxides to obtain energy. For example, iron-reducing bacteria can use reduction of iron oxides to obtain energy. For example, sulfideogenic bacteria (sulfate-reducing bacteria) can use sulfate reduction to obtain energy.

For remediation of compounds that do not adequately aerobically biodegrade, but do anaerobically degrade, peroxides are not added. Anaerobic microorganisms can break down partially oxidized contaminant that is a product of treatment of contaminant with a primary oxidant such as sodium persulfate, for example, as in the S-ISCO™ process. The growth and activity of such anaerobic microorganisms is promoted by a reductive environment. Such a reducing environment can be found, for example, in a region removed from where the primary oxidant is injected. Partially oxidized contaminant can move to a region with a reducing environment, for example, by diffusion or by convection with groundwater. The partially oxidized contaminant itself can consume available oxygen to induce a reducing environment. Furthermore, the partially oxidized hydrocarbon can serve as a food source for anaerobic microorganisms. Alternatively, a microorganism organic food source can be introduced to the subsurface. For example, the microorganism organic food source can be introduced downgradient of the point where the primary oxidant is introduced. For example, the dissolved carbon (food for microorganisms) from VeruSOL, other surfactants, or another substance consumes oxygen, can create an anaerobic environment, and can create a reducing environment enabling processes such as reductive dechlorination to take place. Sulfate, for example, from persulfate, can be reduced biologically so that the redox potential is lowered and reductive dechlorination reactions can take place. Additionally, sulfate can be an alternative electron acceptor to oxygen to enable anaerobic degradation of some petroleum hydrocarbons, such as polycyclic aromatic hydrocarbon (PAH) compounds, such as can be found at manufactured gas plant sites.

Another condition at a site that can favor an approach using bioremediation by anaerobic microorganisms is the presence of chromium(+VI) (hexavalent chromium). Chromium(+VI) is soluble and mobile in the subsurface and is very toxic. On the other hand chromium(+III) is not very soluble and not very mobile in the subsurface and is not very toxic. A reducing environment can reduce the very hazardous chromium(+VI) to the much less hazardous chromium(+III).

To induce a downgradient reductive environment, a food for aerobic bacteria can be injected downgradient of the primary oxidant. The aerobic bacteria can then be made to consume all of the available oxygen to create a reductive environment.

For example, anaerobic microorganisms can metabolize and consume excess surfactant, cosolvent or another substance introduced that migrates away from the locus where it is injected. Thus, following a remediation procedure or outside of a region where remediation is to take place, anaerobic microorganisms can reduce the amount or concentration of excess surfactant, cosolvent or another substance introduced during the remediation process to a predetermined level, to ensure that such an introduced substance is not itself viewed as a contaminant. For example, a predetermined level of an introduced substance can be a maximum amount of an introduced substance at a site permitted by law or can be a maximum concentration of an introduced substance permitted by law.

A reducing zone can be established, for example, by limiting the amount of primary oxidant introduced, introducing a substance that reacts with oxygen, or introducing food for microorganisms. A substance that reacts with oxygen can include a zero-valent metal, zero-valent iron, zero-valent manganese, zero-valent cobalt, zero-valent palladium, zero-valent silver, and a particle of a zero-valent metal coated with a polymer.

Herein, a “reductive zone” and a “reducing environment” refer not only to a zone or environment in which the oxidation potential is less than zero, but also to a zone or environment in which the oxidation potential is equal to or greater than zero, but less than an oxidation potential that is optimal for the growth and metabolism of aerobic microorganisms. That is a “reductive zone” and a “reducing environment” can favor certain anaerobic microorganisms over aerobic microorganisms. In this text, it is convenient to refer to “oxidative zones” and “reductive zones”; however, it is to be understood that there is in fact a continuum of oxidative potentials at a site, for example, a subsurface, to be or being remediated. Thus, in part of an “oxidative zone”, only aerobic microorganisms and no anaerobic microorganisms may be able to proliferate and metabolize, while in another part of the “oxidative zone”, some anaerobic as well as aerobic microorganisms may be able to proliferate and metabolize. In part of a “reductive zone”, only anaerobic microorganisms and no aerobic microorganisms may be able to proliferate and metabolize, while in another part of the “reductive zone”, some aerobic as well as anaerobic microorganisms may be able to proliferate and metabolize.

### Use of Zero-Valent-Metal Particles

Zero valent metal particles can be used in controlling aspects of a remediation process, such as in establishing oxidative zones and/or reductive zones and controlling the rate at which reactions occur in these zones.

For example, zero-valent iron (ZVI) can activate hydrogen peroxide for pentachlorophenol (PCP) and methyl-tertiary-butyl ether (MTBE) destruction. For example, powdered ZVI can activate hydrogen peroxide and increase the rate and extent of compound destruction. However, the rapid reaction rates of ZVI and the rapid oxidation of ZVI with hydrogen peroxide may constraint the direct application of uncoated ZVI particles as activators in subsurface applications.

Nano-scale ZVI (zero-valent-iron) can be injected, e.g., into a subsurface, in an aqueous slurry. The ZVI can be mixed with an organic guar material to provide structural integrity for emplacement into fractures or permeable reactive barriers (PRBs). The ZVI can be mixed with molasses or another type of biodegradable substrate to promote physical, chemical, and/or biological reduction processes, which can occur simultaneously. When organic guar is used with ZVI, an enzyme can be added once the guar-ZVI has been emplaced to induce biodegradation of the guar, thus exposing the ZVI to contaminated groundwater.

In an embodiment, nano-scale (or larger-sized micro-sized powdered) zero valent iron (ZVI) particles can be coated with thin polymer films, and the coated ZVI particles can be introduced into a subsurface to provide sustained activation of persulfate and other oxidants for in situ chemical oxidation. The ZVI-polymer combination can be referred to as “ZVIP” or, alternatively, “poly ZVI.” Such a polymer coating allows for control of the rate of release of a metal activator so as to retain the activator in the subsurface for as long as the oxidant is retained. Thus, the polymer coating can prolong the activation and concentration of the activator. As used herein, “zero valent” means an oxidation state of zero, i.e., without charge. As used herein, “oxidant” includes, for example, persulfate.

Polymers that can be used to coat the ZVI particles include, for example, the biopolymers xanthan polysaccharide, polyglucomannan polysaccharide, emulsan, alginate biopolymers, hydroxypropyl methylcellulose, carboxy-methyl cellulose, ethyl cellulose, chitin, chitosan, as well as, for example, the synthetic polymers polymethyl methacrylate, polystyrene, and polyurethane.

Other zero valent metals can be substituted for zero valent iron (ZVI). For example, zero valent manganese (ZVMn) can be substituted for zero valent iron in every embodiment discussed herein, as can other transition metals, such as cobalt, palladium, and silver. Furthermore, the zero valent metal, for example ZVI and/or ZVMn, can be associated with additional metals, such as palladium and cobalt, as well as other transition metals, for example those that can activate persulfate or increase the reactivity of ZVI, ZVMn, or another zero valent metal.

In an embodiment, nano- and micro-sized coated ZVI particles can be used for sustained activation of Fenton chemistry and persulfate in the destruction of organic and inorganic contaminants in above- and below-ground remediation systems, water supply and waste water treatment systems, as well as industrial applications of Fenton's chemistry and activated persulfate, such as polymer initiators. As used herein, “remediation” means the improvement of the environmental quality of a location, whether such improvement is necessitated by the conduct of humans or otherwise. Drinking water treatment includes processing and treatment of surface and subsurface water to supply potable water whether the systems are large public water supply systems, individual home treatment units, or for treatment of individual wells.

In an embodiment, Fenton chemistry and persulfate activated with polymer coated ZVI particles is used to treat a wide range of organic compounds in industrial wastewaters. For example, organic compounds can include solvents, pesticides, herbicides, polychlorinated biphenyls, dioxin, fuel oxygenates, manufactured gas plants residuals, petroleum derived compounds, semivolatile compounds, chlorinated organic compounds, halogenated organic compounds, and other organic compounds. Polymer coated ZVI particles can be used for sustained activation of Fenton Chemistry and persulfate in industrial waste treatment systems. As used herein, “nano-sized” and “nano-scale” mean particles less than about 1 micron in diameter. As used herein, “micro-sized” and “micro-scale” mean particles from about 1 to about 1000 microns in diameter. As used herein, “macro-sized” and “macro-scale” mean particles greater than about 1000 microns in diameter. In an embodiment, permeable reactive barriers can be constructed that contain mixtures of macro-, micro- and/or nano-sized PolyZVI particles with other, more mobile reductants, and can be used for remediation. As used herein, “reductants” includes the organic compounds listed above as well as several inorganic compounds, such as perchlorate, chromate, and arsenic. Examples of additional reductants are molasses, vegetable oils, and other plant-based organic chemicals, such as VeruSOL.

In an embodiment, the polymer coated ZVI or ZVMn particles can be emulsified using various surfactants enabling: 1) less reaction with subsurface materials and greater transport distance in the subsurface, 2) mixing with other S-ISCO reagents during preparation and injection into the subsurface, 3) coelution with S-ISCO reagents such as cosolvents, surfactants, and oxidants or any combination thereof, 4) intimate contact in micelles with dissolved and emulsified NAPLs, better penetration into the subsurface for use as a reduction technology, and 5) greater compatibility and effectiveness for mixing ZVI and ZVMn with surfactants and plant based biologically degradable amendments, such as vegetable oils and molasses.

Polymer coated particles, such as polymer coated zero valent metal particles, can be used with a surfactant or mixture of cosolvents and surfactants to emulsify the polymer coated particles. The particles, together with solubilizing or emulsifying plant oils or other plant extracts can be injected into the subsurface or spread on a ground surface to create a zone combining physical, chemical, and/or biological processes, or any combination thereof to reductively destroy or transform organic or inorganic contaminants in the environment. The biological processes can involve, for example, anaerobic organisms. As used herein, “blending” includes, for example, emulsifying, solubilizing, or a combination thereof.

As used herein, “reducing agent” includes, for example, zero valent metal particles. As used herein, “half-life” means the time required for one half of a process to be completed, for example the time required for one half of a reagent to be consumed during a reaction.

Nanoscale and microscale ZVI particles (and/or other zero valent metal particles) can be injected into the subsurface to create reduction zones by, for example: 1) creating a matrix in which the ZVI is emplaced that allows transport of the ZVI into the subsurface with a minimum amount of reaction of the ZVI particles with, or adhesion of the ZVI particles to, the subsurface mineralogy, for example, to prevent clogging and to promote penetration of the ZVI particles through the subsurface soil; and/or 2) adding a biological substrate such as molasses or vegetable oil, which can provide a longer term biologically created reduction zone than that achievable with nanoscale or microscale ZVI alone.

A goal of in situ remediation using chemical oxidation is to maximize the volume of soil treated for each injection well or injection location. A factor affecting the volume of soil treated is the longevity of the oxidant in the subsurface, given injection flow rates, oxidant concentration, volume of injected fluid, mass of oxidant added and the type of activation system used. When activation is required, such as in the case of oxidants such as persulfate and peroxide, the longevity of the activator should ideally be close to that of the oxidant. To date, no activation system has been demonstrated to have as long a life in the subsurface as persulfate. Polymer coated ZVI (and/or other zero valent metal) particles according to an aspect of the invention can enable a more controlled time-release of the metal activator and/or a more controlled rate of activation of oxidants such as persulfate and peroxide than is possible with previously known metal-chelate systems.

Zero valent metal particles can be used to activate an oxidant, such as persulfate and/or hydrogen peroxide for in situ chemical oxidation remediation. Nano- or larger-scale zero valent metal (e.g., ZVI) material coated with, for example, partitioning polymers, surfactant materials, or electrically conducting polymers can be applied in the remediation of dense non-aqueous phase liquid (DNAPL), light non-aqueous phase liquid (LNAPL), and other contaminants and chemicals of concern.

Aspects of the invention also can be employed in contexts other than treatment and remediation. For example, an aspect of the invention can be utilized in the air purification, water supply, and drug therapy fields. Production chemical and polymer activation can be novel applications of coated Zero-Valent Iron (ZVI) and/or other zero valent metals.

For example, polymer coating of macro-sized ZVI (and/or other zero valent metal) particles, which can be used in permeable reactive barriers (PRBs), can extend the life of these barriers, e.g., by a factor or two or more. For example, resistant polymer coatings can be added to the ZVI particles that can be removed by some specialized chemistry, such as an acid or base rinse, chemical stripping, thermal or electrical processes, or certain biological processes. As used herein, “removing agent” means compounds or other substances that possess the ability to remove the polymer coating from a zero valent iron particle or other activator particle. Mixtures of polymer coated ZVI particles with non-polymer coated particles can be used in PRBs. For example, once non-coated ZVI particles have been exhausted and are no longer able to sufficiently reduce target contaminants (e.g., after a 20 year life), then the polymer coated ZVI particles can be treated in situ to remove the coatings and obtain a second 20 year period of PRB life.

Polymer coated ZVI (and/or other zero valent metals) can be used to create hydrophobic particles for remediation, for example for DNAPL partitioning. ZVI particles can be coated with multiple polymer layers. For example, the polymer layers can be of varying polymer type and polymer layer thickness. A set of ZVI particles having different thicknesses of polymer coating can be used in remediation. The distribution of polymer coating thicknesses can be tailored to control the rate at which the ZVI particles activate oxidant or reduce contaminants and/or other compounds in the environment at various times in the remediation process. One purpose of a polymer coated ZVI can be to act as an activator of an oxidant; in such an application it can be important that the polymer coated ZVI remains capable of activating the oxidant for as long as there is oxidant to be activated in the location. It can be important that the activator is not consumed too rapidly. For example, the polymer coating can limit the rate at which the activator is exposed to the oxidant.

The activator should travel as far as the oxidant in the location to be remediated. An uncoated activator may adhere to soil particles, which would inhibit the activator's migration through the location. By applying a polymer coating to the activator surface, activator adherence to soil particles can be minimized.

Altering the polarity and electrolytic conductivity of the polymer can enable control of sorptive processes, including potential partitioning into LNAPL and DNAPL mixtures. For example, the following factors can be adjusted in applying poly-ZVI (and/or polymer coated particles of other zero valent metals) to in situ applications: (i) the type of polymer used, based on its reactivity with an oxidant such as persulfate, biodegradation of by-products and ability of ZVI to be coated; (ii) the thickness of the coating, to control the rate at which the zero valent iron is exposed to oxidants such as persulfate resulting in a controlled rate of free radical production; (iii) the molecular size and other physical properties of the polymer, to control penetration of the polymer into the nano-scale ZVI; (iv) the electrolytic conductivity of the coating, to avoid or induce sorption of the ZVIP onto various mineral and organic materials, as appropriate for remediation applications; and (v) the polarity of the polymer coating, to control adsorptive and absorptive partitioning of the ZVIP with natural organic carbon, bacterial mass, and/or LNAPL and DNAPL.

### Design of Combined S-ISCO™ and Biodegradation Remediation Processes

A method for determining a contaminant remediation protocol, for example, of a protocol for remediating soil in a subsurface contaminated with NAPL or other organic chemicals, can include the following steps. Site soil samples can be collected under zero headspace conditions (e.g., if volatile chemicals are present); for example, samples representative of the most highly contaminated soils can be collected. The samples can be homogenized for further analysis. A target contaminant or target contaminants in the soil can be identified. The demand of a sample of oxidant per unit soil mass can be determined; for example, the demand of a soil sample for a persulfate oxidant, such as sodium persulfate, can be determined. An oxidant is, for example, a chemical or agent that removes electrons from a compound or element, increases the valence state of an element, or takes away hydrogen by the addition of oxygen. A suitable oxidant and/or a suitable mixture of an oxidant and an activator for oxidizing the target contaminant can be selected. The oxidant can include an oxidant that generates a gas phase upon its decomposition in the subsurface, the oxidant can be added as a gas, or the oxidant can be added as a dissolved gas. Further, in addition to the added oxidant, dissolved gas under pressure can be added to the subsurface to generate a gas phase. The behavior of the gas phase, in addition to the cosolvent-surfactant mixture or surfactant alone, can lead to enhanced extraction of the contaminant. Further, a dissolved gas under pressure can be added to the subsurface to generate a gas phase in addition to a cosolvent-surfactant mixture or surfactant, which leads to enhanced extraction of the contaminant. Suitable surfactants, mixtures of surfactants, and/or mixtures of surfactants, cosolvents, and/or solvents capable of solubilizing and/or desorbing the target contaminant or contaminants can be identified; for example, suitable biodegradable surfactants can be tested. Suitable solvents capable of solubilizing and/or desorbing the target contaminant or contaminants can be identified; for example, suitable biodegradable solvents such as d-limonene can be tested. Various concentrations of cosolvent-surfactant mixtures or surfactants alone can be added to water or groundwater from a site along with controlled quantities of NAPLs. Relationships of the extent of dissolution of the NAPL compounds with the varying concentrations of the cosolvent-surfactant mixtures or surfactants can be established by measuring the concentrations of the NAPL compounds that enter the aqueous phase. Relationships between the interfacial tension and solubilized NAPL compounds and their molecular properties, such as the octanol-water partition coefficient (K_{ow}) can also be established that enable optimal design of the dissolution portion of the S-ISCO process. Various concentrations of cosolvent-surfactant mixtures or surfactants alone can be added to water or groundwater from a site along with controlled quantities of contaminated soils from the site. Relationships of the extent of solubilization of the sorbed COC compounds with the varying concentrations of the cosolvent-surfactant mixtures or surfactants can be established by measuring the concentrations of the sorbed COCs that enter the aqueous phase. Relationships between the interfacial tension and desorbed and solubilized compounds and their molecular properties, such as the octanol water partition coefficient (K_{ow}), can also be established that enable optimal design of the dissolution portion of the S-ISCO process. The simultaneous use of oxidants and surfactants or cosolvent-surfactant mixtures in decontaminating soil can be tested. For example, the effect of the oxidant on the solubilization characteristics of the surfactant can be evaluated, to ensure that the oxidant and surfactant can function together to solubilize and oxidize the contaminant. The quantity of surfactant for injection into the subsurface can be chosen to form a Winsor I system or a microemulsion.

For example, the type and quantity of surfactants and optionally of cosolvent required to solubilize the target contaminant can be determined in a batch experiment.

The spatial concentration distribution of the target contaminant can be determined. A hydrogeological property of the subsurface can be determined. The determined spatial concentration distribution of the target contaminant and the hydrogeological property can be used to determine a target depth for the oxidant, gas phase generating oxidant, or pressurized dissolved gas in liquid, cosolvent-surfactant or surfactant and injection site(s) of the above injectants, and an extraction site for the contaminant.

Experimentation on the effects of various oxidants, combinations of oxidants, and activators on the stability and activity of cosolvent-surfactant mixtures and surfactants can be readily conducted to provide information to optimize S-ISCO treatment conditions. Testing of the sorption or reaction of the surfactant or surfactant-cosolvent mixture can be conducted to determine the transport and fate properties of the surfactant or surfactant-cosolvent mixture in soils, rock and groundwater. Testing can be conducted in batch aqueous or soil slurry tests in which individual cosolvent-surfactant mixtures or surfactants at specified initial concentrations are mixed together with individual oxidants or mixtures of oxidants and activators. The duration of the tests can be a minimum of 10 days and as long as 120 days, dependent on the stability of the oxidant-surfactant system needed for a particular application.

Testing of oxidants, surfactants, activators, cosolvents, and/or solvents can be conducted with the contaminant in the non-aqueous phase and/or sorbed phase in aqueous solution, or with the contaminant in a soil slurry or soil column. A soil slurry or soil column can use a standard soil or actual soil from a contaminated site. An actual soil can be homogenized for use in a soil slurry or soil column. Alternatively, an intact soil core obtained from a contaminated site can be used in closely simulating the effect of introduction of oxidant, surfactant, and/or solvent for treatment.

The microorganism population in a site to be remediated, for example, a subsurface, can be characterized. For example, a technique such as metagenomic analysis or another technique can be used to determine the types and density (e.g., number per unit subsurface volume) of aerobic and/or anaerobic microorganisms at one or more locations in a site to be remediated. For example, resident microorganisms such as bacteria, fungi, molds, yeasts, archaea, protists, algae, plankton, microscopic plants, protozoans, lichens, microscopic animals, planaria, and amoebas can be identified. Maps of the site to be remediated that present concentrations of different microorganisms can be developed and can be used in the development of a remediation process. Microorganisms that are desirable, for example, because they biodegrade contaminants or partially oxidized products of contaminants and/or because they biodegrade excess oxidant, surfactant, and/or cosolvent, can be identified. Conditions which cause such desirable microorganisms to proliferate and/or increase the metabolic rate of such desirable microorganisms can be identified either through applying prior knowledge or conducting experiments. Microorganisms not present in the site to be remediated, but would be desirable if present can be identified, and protocols for seeding the site to be remediated with such desirable microorganisms can be developed.

The effect of oxidants, surfactants, activators, cosolvents, and/or solvents to be used in the remediation process on aerobic and anaerobic microorganisms can be tested. The selection of substances such as oxidants, surfactants, activators, cosolvents, and/or solvents for the remediation process can be indicated by the tendency of a substance, alone or in conjunction with another substance, to promote the proliferation and/or metabolism of desirable microorganisms and/or to suppress the proliferation and/or metabolism of undesirable microorganisms. The selection of a substance can be contraindicated by its tendency to promote the proliferation and/or metabolism of undesirable microorganisms and/or to suppress the proliferation and/or metabolism of desirable microorganisms. Such testing can indicate the introduction of additional substances to the site to be remediated for the purpose of promoting the proliferation and/or metabolism of desirable microorganisms and/or suppressing the proliferation and/or metabolism of undesirable microorganisms. For example, such testing can indicate that nutrients for desirable microorganisms be added, that acid, base, and or buffer be added to establish and maintain an optimal pH for desirable microorganisms, and/or that an oxidant or reductant be added to a particular region of the site to be remediated, for example, to establish an oxidative or reductive zone.

### Monitoring and Control of Combined S-ISCO™ and Biodegradation Remediation Processes

Before, during, and after the injection of injection fluid and the removal of contaminant, the subsurface can be monitored to ensure that the remediation process is proceeding satisfactorily.

A method can include monitoring the concentration and/or spatial distribution of quantities such as contaminant, products of partial oxidation of contaminant, oxidant, reductant, surfactant, cosolvent, and/or aerobic and/or anaerobic microorganisms in a site being remediated, such as a subsurface. Examples of other quantities that can be monitored include pH, oxidation potential, temperature, pressure, salinity, and concentration of other substances. Monitoring can be conducted at one or several locations within the site being remediated. The monitoring date can be used to monitor the progress of the remediation, for example, reduction in the concentration of contaminant. The monitoring data can be used to detect, for the purpose of suppressing, undesired conditions, such as the migration of contaminant off of a site being remediated. The monitoring data can be used in determining how to achieve goals of the remediation, for example, speedy destruction of a contaminant without mobilization of the contaminant off of a site. For example, the monitoring data may indicate that the introduction of substances such as oxidants, reductants, surfactants, activators, cosolvents, solvents, nutrients, and/or seeded microorganisms indicated (or contraindicated) during the design phase of a project be stopped or started. For example, the monitoring data may indicate that parameters such as concentration and/or flow rate of an injected substance and/or vacuum imposed on an extraction well be changed. The monitoring data can be provided to a control system that can automatically adjust parameters of the remediation process.

For example, the concentration and/or spatial distribution of hydrogen peroxide, another oxidant, a surfactant, and/or a cosolvent that have been injected into the subsurface can be monitored continuously, periodically, or sporadically, for example, to ensure that the hydrogen peroxide, another oxidant, a surfactant, and/or a cosolvent are being transported to regions of the subsurface where they can promote the oxidation of contaminant and mobilization of contaminant to an extraction well. For example, the concentration and/or spatial distribution of the contaminant, one or more components of the contaminant, the product of contaminant oxidation, and/or one or more components of the product of contaminant oxidation can be monitored continuously, periodically, or sporadically. For example, such monitoring can ensure that the contaminant is being destroyed by oxidation, modified by oxidation, so that it is more susceptible to degradation, e.g., by a chemical or microbial process, being mobilized to an extraction well, and/or not being mobilized to a region in which the contaminant can have a more deleterious impact, e.g., below a residential area, than the region where the contaminant was located prior to starting remediation.

### EXAMPLES

### Example 1

A method for reducing the concentration of a contaminant in a soil includes the following. An oxidant and a surfactant can be introduced into a subsurface containing the soil. The surfactant can solubilize and/or desorb the contaminant. The oxidant can oxidize the solubilized contaminant in the subsurface, so that the amount of the contaminant in the soil is substantially reduced. A microorganism in the subsurface can biodegrade the contaminant and/or products of oxidation of the contaminant. The overall rate of oxidization of the contaminant can be controlled to a predetermined value and the overall rate of solubilization of the contaminant can be controlled to a predetermined value by selecting the oxidant, surfactant, and antioxidant and adjusting the concentrations of surfactants, oxidants, and antioxidants, so that the rate of oxidation of the contaminant is greater than, less than, or equal to the rate of solubilization of the contaminant in accordance with a predetermined decision.

### Example 2

In an embodiment, a composition includes soil, a non-aqueous phase contaminant, a quantity of surfactant, an oxidant, and a microorganism. The quantity of surfactant can be sufficient to solubilize the nonaqueous phase liquid contaminant. The surfactant can form a Windsor I solution or microemulsion. The microorganism can biodegrade the contaminant and/or products of oxidation of the contaminant.

### Example 3

In an embodiment, a treated composition includes soil, an oxidized contaminant, and an oxidant residue. The treated composition can further include a microorganism.

### Example 4

A method for reducing the concentration of a contaminant in soil can include solubilizing the contaminant and oxidizing the contaminant. Mobilization of the contaminant during solubilization and oxidation can be minimal. The method can further include using a microorganism to biodegrade the contaminant and/or products of oxidation of the contaminant.

### Example 5

A method for reducing the concentration of a contaminant in soil can include locally mobilizing the contaminant and oxidizing the contaminant. The method can further include using a microorganism to biodegrade the contaminant and/or products of oxidation of the contaminant.

### Example 6

A method for determining a subsurface contaminant remediation protocol can include the following. A soil sample can be collected from the subsurface. At least one target contaminant for concentration reduction can be identified. A surfactant and/or a cosolvent can be chosen for injection into the subsurface to solubilize the at least one target contaminant. An oxidant and optionally an activator for the oxidant can be chosen for injection into the subsurface to oxidize the target contaminant. A quantity of surfactant can be chosen for injection into the subsurface to form a Winsor I system or a submicellar surfactant solution or a microemulsion. A microorganism or class of microorganisms can be selected to biodegrade the identified contaminant and/or products of oxidation of the identified contaminant.

### Example 7

A method for determining a subsurface contaminant remediation protocol can include the following. A soil sample from the subsurface can be collected. At least one target contaminant for concentration reduction can be identified. A surfactant or surfactants and/or a solvent can be chosen for injection into the subsurface to desorb and solubilize the at least one target contaminant. An oxidant and optionally an activator can be chosen for injection into the subsurface to oxidize the target contaminant. A quantity of surfactant for injection into the subsurface to form a Winsor I system or a microemulsion can be chosen. The spatial concentration distribution of the target contaminant can be chosen. A hydrogeological property of the subsurface can be determined. The determined spatial concentration distribution of the target contaminant and the hydrogeological property can be used to determine the target depth for the surfactant and oxidant and optionally for the solvent and activator. A microorganism or class of microorganisms can be selected to biodegrade the identified contaminant and/or products of oxidation of the identified contaminant.

### Example 8

A method for reducing the concentration of a contaminant in a soil at a target depth can include the following. A target depth range for reducing the concentration of the contaminant can be identified. A surfactant, an oxidant, and optionally a non-oxidant, non-activator salt can be selected. The surfactant, the oxidant, and optionally the non-oxidant, non-activator salt can be introduced into a subsurface containing the soil. The surfactant can solubilize and/or desorb the contaminant. The oxidant can oxidize the contaminant in the subsurface, so that the concentration of the contaminant in the soil is substantially reduced. A microorganism or class of microorganisms can be selected to biodegrade the identified contaminant and/or products of oxidation of the identified contaminant. The surfactant and the oxidant can be introduced together and the oxidant can be selected so that the combination of the surfactant and the oxidant has a density to maximize the fraction of the surfactant and oxidant mixture that remains within the target depth range. The non-oxidant, non-activator salt can be introduced together with the surfactant, the oxidant, or both, and the non-oxidant, non-activator salt can be selected so that the mixture of the non-oxidant, non-activator salt with the surfactant, the oxidant, or both has a density to maximize the fraction of the surfactant and maximize the fraction of the oxidant that remains within the target depth range.

### Example 9

A method for reducing the initial mass of a contaminant in a volume of soil can include the following. A volume of a solution including an oxidant and a volume of a solution including a surfactant can be introduced into a substrate containing the soil. A microorganism or class of microorganisms can be selected to biodegrade the identified contaminant and/or products of oxidation of the contaminant. At least 40% of the initial mass of contaminant can be eliminated from the volume of soil. In an embodiment, no more than 5% of the combined volume of the solution including the oxidant and the volume of the solution including the surfactant is extracted from the soil.

### Example 10

A method for reducing the concentration of a contaminant in a soil can include the following. An oxidant and a surfactant can be introduced into a ground surface or above-ground formation, structure, or container containing the soil. The surfactant can solubilize and/or desorb the contaminant. The oxidant can oxidize the solubilized contaminant, so that the amount of the contaminant in the soil is substantially reduced. A microorganism or class of microorganisms can be selected to biodegrade the identified contaminant and/or products of oxidation of the contaminant. The overall rate of oxidization of the contaminant can be controlled to a predetermined value and the overall rate of solubilization of the contaminant can be controlled to a predetermined value by selecting the oxidant, surfactant, and antioxidant and adjusting the concentrations of surfactants, oxidants, and antioxidants, so that the rate of oxidation of the contaminant is greater than, less than, or equal to the rate of solubilization of the contaminant in accordance with a predetermined decision.

### Example 11

### Remediation of Chlorinated Solvent

An embodiment of the invention is the simultaneous or sequential use of cosolvent-surfactant mixtures, for example, Citrus Burst 3 with activated persulfate (activated at a high pH with NaOH) for the treatment of sites contaminated with chlorinated solvents and other chlorinated or halogenated compounds.

In order to test the treatment of chlorinated compounds, a chlorinated solvent DNAPL was obtained from a site consisting of chlorinated solvents and chlorinated semi-volatile compounds. Composition of the chlorinated solvent DNAPL is presented based on determinations using USEPA Methods 8260 and 8270. An aliquot of the DNAPL was mixed with a suitable quantity of deionized water to determine the equilibrium solubility of the individual compounds in the presence of the DNAPL. Experimental conditions for these dissolution tests are reported in Table 2.

TABLE 2
Experimental Conditions for Chlorinated DNAPL Dissolution
Experiments

| | Water | | Citrus | Citrus | | | |
|---|---|---|---|---|---|---|---|
| Exp. | g | DNAPL | Burst-3 | Burst-3 | DNAPLₘₐₓ | NaCl | NaCl |
| No. | Total | g | g | g/L | g/L | g | g/L |
| 1 | 60 | 2 | 0.05 | 0.8 | 33.3 | 3 | 50 |
| 2 | 60 | 2 | 0.1 | 1.7 | 33.3 | 3 | 50 |
| 3 | 60 | 2 | 0.25 | 4.2 | 33.3 | 3 | 50 |
| 4 | 60 | 2 | 0.5 | 8.3 | 33.3 | 3 | 50 |
| 5 | 60 | 2 | 1 | 16.7 | 33.3 | 3 | 50 |
| 6 | 60 | 2 | 2.5 | 41.7 | 33.3 | 3 | 50 |
| 7 | 60 | 2 | 5 | 83.3 | 33.3 | 3 | 50 |
| 8 | 60 | 2 | 0 | 0.0 | 33.3 | 3 | 50 |
| 9 | 60 | 2 | 0 | 0.0 | 33.3 | 3 | 50 |

The data collected under the experimentation conditions presented in Table 2 were obtained at 25° C. with 60 rpm shaker table mixing for 48 hours. After the shaker was shut off, the samples sat quietly for 5 minutes before the supernatant was analyzed. DNAPLₘₐₓ represents the maximum concentration of DNAPL that may dissolve, given the mass of DNAPL and the volume of water.

Results of these analyses and the pure compound solubilities of the individual compounds are reported in Table 3.

TABLE 3
Chlorinated DNAPL Composition and Dissolution in Control Sample
Without Cosolvent-Surfactant

| | | Observed | | Pure Compound |
|---|---|---|---|---|
| | | Solubility in | | Aqueous |
| | DNAPL | Control Sample | DNAPL | Solubility |
| Compound | Composition % | (mg/L) | Mol Fraction | (mg/L) |
| Tetrachloroethene (PCE) | 67.68% | 140 | 0.194 | 800 |
| Carbon Tetrachloride (CTC) | 19.65% | 100 | 0.724 | 129 |
| Hexachlorobutadiene (HCBD) | 4.15% | NA | 0.006 | 0.005 |
| Hexachlorobenzene (HCB) | 0.93% | 1.4 | 0.024 | 3.2 |
| Hexachloroethane (HCE) | 7.42% | NA | 0.051 | 50 |
| Octachlorostyrene (OCS) | 0.16% | NA | 0.000 | insoluble |
| Octachloronaphthalene (OCN) | 0.01% | NA | 0.001 | insoluble |

Carbon tetrachloride and tetrachloroethylene comprised more than 87 percent of the DNAPL. Being a saturated compound, carbon tetrachloride is generally a pervasive and difficult to degrade compound once introduced to the subsurface. The observed solubilities of the DNAPL compounds in the aqueous phase are quite low and will be the basis to compare enhanced dissolution using Citrus Burst-3. After 48 hours of slowly mixing the DNAPL and water mixtures, the samples were allowed to sit for 5 minutes and then samples of the solubilized fraction of the mixture were collected and analyzed for VOCs using USEPA Method 8260. Samples from experiment number 1, 3, 5, 7, and 8 (control) were analyzed. Additionally, measurements of interfacial tension (IFT) were conducted on the samples after the 48-hour period.

Once the concentrations of the VOC compounds in the solubilized phase were measured, the solubility enhancement factors, β, was calculated for each compound at each Citrus Burst concentration. β is the ratio of the concentration in mg/L of the individual VOC compound dissolved with the CB-3 divided by the solubility of the same individual VOC compound dissolved in the presence of the DNAPL without the cosolvent surfactant. The results of this test are found in FIG. 6. The β values varied from a low of 2.79 for carbon tetrachloride at a Citrus Burst concentration of 0.8 g/L, to a high of 857.14 for hexachlorobutadiene at a Citrus Burst concentration of 83.3 g/L. A log-normal plot of the total VOCs dissolved using various doses of Citrus-Burst 3 versus the interfacial tension measurement (IFT) taken in each vial after 48 hours of contact can be found in FIG. 7. For example, it can be readily observed from FIG. 6 that IFT measurements can be used to easily determine the solubility potential of the cosolvent-surfactant mixture. The highly linear log-normal relationship of the logarithm of the octanol-water partition coefficient (log(K_{ow})) and the solubility enhancement factor, β, for each of the tested Citrus Burst-3 concentrations allows prediction of the solubility behavior of many organic compounds using the relationship. These types of experiments and relationships can be used to screen and determine optimal types and concentrations of surfactants and cosolvent-surfactant mixtures that can be used to optimize dissolution of NAPL organic compounds useful in the S-ISCO process.

Aliquots of the Citrus Burst-3 enhanced solubilized DNAPL mixtures were added to aliquots of a sodium persulfate solution and the bulk solution pH adjusted to greater than 12 using NaOH. Prior to adding the sodium persulfate, initial VOC and SVOC concentrations of the solutions were determined using USEPA Methods, 8260 and 8270, respectively, as shown in Table 3. These solutions were slowly mixed at 60 rpm on an orbital shaker table for 14 days. After the 14 day mixing period the solutions were removed from the mixer and the VOC and SVOC concentrations were measured using USEPA Methods 8260 and 8270. The overall removal of VOCs and SVOCs was calculated for each treatment condition and the results can be found in FIG. 8. The T1 and T3 samples, which initially had 0.8 g/L and 4.3 g/L, respectively of Citrus-Burst 3, had greater than 99 percent removals of VOCs and SVOCs after 14 days of treatment. The T7 sample that initially had a Citrus Burst-3 concentration 83.3 g/L and a much greater concentration of VOCs and SVOCs than the other vials, removed of VOCs and SVOCs were 94 percent and 76 percent, respectively. The initial IFT measurements for the T1, T3, and T7 tests prior to oxidation were 63.9 mN/m, 48.5 mN/m and 35.40 mN/m, respectively. Following the 14 day oxidation period, the final IFT readings for the T1, T3, and T7 tests were 74.4 mN/m, 73.1 mN/m and 35.40 mN/m, respectively. The alkaline persulfate substantially removed the dissolved VOCs and SVOCs from the T1 and T3 samples, as well as returning the IFT values to background conditions of water without any added cosolvent-surfactant. In the case of the T7 sample, the IFT values remained low while high removal percentages of the VOCs and SVOCs were observed. It is likely that additional time was required to destroy the remaining VOCs and SVOCs in the T7 vial and to increase the IFT to background conditions. Digital photographs were taken of the test vials before, during and after the 14 day treatment. It was evident after 14 days of treatment that the turbidity and red color (associated with the Suidan IV dyed DNAPL) were completely removed and the solutions returned to a clear condition. In the T7 sample, the red color was removed (indicative of most of the dissolved DNAPL removed) and much of the turbidity was reduced.

Additional tests that can be performed include the ability of various microorganisms to biodegrade the chlorinated hydrocarbons and/or partially oxidized products of the chlorinated hydrocarbons. Further, tests can be performed on how to optimize conditions such as pH and oxidation potential to promote the proliferation and metabolism of microorganisms that can perform such biodegradation.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

1. A method for reducing the concentration of a contaminant in a subsurface to a predetermined level, comprising:
introducing a primary oxidant and a surfactant and/or a cosolvent into the subsurface;
the surfactant solubilizing or desorbing the contaminant;
the primary oxidant oxidizing the solubilized contaminant in the subsurface to a biodegradable compound; and
a microorganism in the subsurface biodegrading the biodegradable compound, so that the concentration of the contaminant in the subsurface is reduced to the predetermined level.

2. (canceled)

3. The method of claim 1, further comprising:
monitoring the subsurface for a quantity selected from the group consisting of contaminant concentration, oxidant concentration, surfactant concentration, cosolvent concentration, microorganism concentration, and combinations; and
adjusting the amount of oxidant, surfactant, and/or cosolvent introduced into the subsurface to minimize the amount of contaminant in the subsurface.

4. The method of claim 1,
wherein the primary oxidant establishes an oxidative zone in the subsurface in the vicinity of the locus of introduction of the primary oxidant,
wherein aerobic microorganisms proliferate in the oxidative zone and biodegrade the biodegradable compound and/or the contaminant,
wherein oxidation of the solubilized contaminant and/or biodegradation of the biodegradable compound by the microorganism consumes the primary oxidant and establishes a reductive zone in the subsurface surrounding the oxidative zone,
wherein anaerobic microorganisms proliferate in the reductive zone and biodegrade the biodegradable compound and/or the contaminant.

5. The method of claim 4,
further comprising introducing a secondary oxidant in the subsurface in the oxidative zone and downgradient of the locus of introduction of the primary oxidant,
wherein the oxidative zone extends downgradient from the locus of introduction of the primary oxidant,
wherein the reductive zone extends farther downgradient from the locus of introduction of the primary oxidant than does the oxidative zone and
wherein introducing the secondary oxidant stimulates the proliferation of, the metabolism of, and/or the biodegradation of the biodegradable compound and/or the contaminant by the aerobic microorganisms.

6. (canceled)

7. The method of claim 4, further comprising
introducing a reductant in the subsurface downgradient of the locus of introduction of the primary oxidant,
wherein introducing the reductant stimulates the proliferation of, the metabolism of, and/or the biodegradation of the biodegradable compound and/or the contaminant by the anaerobic microorganisms,
wherein the reductant induces a reductive potential in the reductive zone by reacting with oxygen in the subsurface.

8. (canceled)

9. The method of claim 4, further comprising
introducing a reductant in the subsurface downgradient of the locus of introduction of the primary oxidant,
wherein introducing the reductant stimulates the proliferation of, the metabolism of, and/or the biodegradation of the biodegradable compound and/or the contaminant by the anaerobic microorganisms and
wherein the reductant induces a reductive potential in the reductive zone by acting as a food source for aerobic microorganisms and thereby stimulates the consumption of oxygen in the subsurface by the aerobic microorganisms.

10.-11. (canceled)

12. The method of claim 1, further comprising pumping contaminant out of an extraction well to establish an extraction zone in the subsurface.

13. (canceled)

14. The method of claim 1, wherein the overall rate of oxidization of the contaminant is controlled to a predetermined value and the overall rate of solubilization of the contaminant is controlled to a predetermined value by selecting the primary oxidant and surfactant and adjusting the concentration of primary oxidants and surfactants, so that the rate of oxidation of the contaminant is greater than, less than, or equal to the rate of solubilization of the contaminant in accordance with a predetermined decision.

15.-16. (canceled)

17. The method of claim 1, further comprising introducing a microorganism into the subsurface downgradient of the point where the oxidant, surfactant, and/or cosolvent is introduced.

18. (canceled)

19. The method of claim 1, wherein the microorganism is an aerobic microorganism and wherein proliferation of the microorganism is stimulated by inducing an oxidative subsurface environment by introducing the primary oxidant.

20.-21. (canceled)

22. The method of claim 1, wherein the microorganism is an anaerobic microorganism and wherein proliferation of the microorganism is stimulated by inducing a reductive subsurface environment by limiting the amount of primary oxidant introduced, introducing a substance that reacts with oxygen, or introducing food for microorganisms.

23. The method of claim 22,
wherein the substance that reacts with oxygen is selected from the group consisting of a zero-valent metal, zero-valent iron, zero-valent manganese, zero-valent cobalt, zero-valent palladium, zero-valent silver, and a particle of a zero-valent metal coated with a polymer and
wherein the polymer is selected from the group consisting of xanthan polysaccharide, polyglucomannan polysaccharide, emulsan, an alginate biopolymer, hydroxypropyl methylcellulose, carboxy-methyl cellulose, ethyl cellulose, chitin, chitosan, polymethyl methacrylate, polystyrene, and polyurethane.

24.-25. (canceled)

26. The method of claim 1, further comprising introducing a nutrient into the subsurface, wherein the nutrient is selected from a carbon source, a phosphorous source, a nitrogen source, a sulfur source, a potassium source, a sodium source, an iron source, and a magnesium source.

27. The method of claim 1, further comprising introducing a nutrient into the subsurface, wherein the nutrient promotes proliferation of aerobic microorganisms and biodegradation of the biodegradable compound.

28. The method of claim 1, further comprising introducing a nutrient into the subsurface, wherein the nutrient promotes proliferation of anaerobic microorganisms and biodegradation of the biodegradable compound.

29. (canceled)

30. The method of claim 1, wherein the primary oxidant is selected from the group consisting of potassium persulfate, ammonium persulfate, and potassium permanganate.

31. The method of claim 1, wherein the surfactant and/or cosolvent comprises VeruSOL surfactant.

32. (canceled)

33. The method of claim 1, wherein the surfactant and/or cosolvent is selected from the group consisting of a carboxylate ester, a plant-based ester, a terpene, a citrus-derived terpene, limonene, d-limonene, castor oil, cocoa oil, cocoa butterₛ coconut oil, soy oil, tallow oil, cotton seed oil, a naturally occurring plant oil, a plant extract, a nonionic surfactant, ethoxylated soybean oil, ethoxylated castor oil, ethoxylated coconut fatty acid, amidified, ethoxylated coconut fatty acid, and combinations.

34.-35. (canceled)

36. The method of claim 1, wherein the surfactant and/or cosolvent is selected from the group consisting of ALFOTERRA 123-8S, ALFOTERRA 145-8S, ALFOTERRA L167-7S, ETHOX HCO-5, ETHOX HCO-25, ETHOX CO-40, ETHOX ML-5, ETHAL LA-4, AG-6202, AG-6206, ETHOX CO-36, ETHOX CO-81, ETHOX CO-25, ETHOX TO-16, ETHSORBOX L-20, ETHOX MO-14, S-MAZ 80K, T-MAZ 60 K 60, TERGITOL L-64, DOWFAX 8390, ALFOTERRA L167-4S, ALFOTERRA L123-4S, ALFOTERRA L145-4S.

37. (canceled)

38. The method of claim 1, further comprising introducing an activator into the subsurface, wherein the activator is selected from the group consisting of a metal activator, a chelated metal activator, a chelated iron activator, Fe(II)-EDTA, Fe(III)-EDTA, Fe(II)-citric acid, Fe(III)-citric acid, and Fe-NTA.

39. (canceled)

40. The method of claim 38,
wherein the activator has the form of a particle coated with a polymer selected from the group consisting of xanthan polysaccharide, polyglucomannan polysaccharide, emulsan, an alginate biopolymer, hydroxypropyl methylcellulose, carboxy-methyl cellulose, ethyl cellulose, chitin, chitosan, polymethyl methacrylate, polystyrene, and polyurethane,
wherein the polymer coating is sufficiently thick for the activator to remain capable of activating an oxidant in a location in need of remediation for at least as long as an oxidant capable of oxidizing contaminant in the location remains in the location and
wherein the polymer coating is permeable to an atomic or molecular species selected from the group consisting of persulfate, sulfate, peroxide, hydroperoxide, oxygen, and hydroxyl.

41.-42. (canceled)

43. The method of claim 40, wherein the activator particle travels with the primary oxidant.

44. The method of claim 1, further comprising introducing an antioxidant into the subsurface.

45. The method of claim 1, wherein an oxidizing environment is established in a region of the subsurface around where the primary oxidant is introduced and wherein a reducing environment is established in a region downgradient of the oxidizing environment and/or surrounding the oxidizing environment.

46. The method of claim 1, further comprising introducing a compound that reacts with oxygen or introducing food for microorganisms into the subsurface away from or downgradient of where the primary oxidant is introduced.

47. The method of claim 1, wherein a reducing environment is established in a region of the subsurface, and wherein an oxidizing environment is established in a region downgradient of the reducing environment and/or surrounding the reducing environment by introducing the primary oxidant and or introducing hydrogen peroxide.

48. The method of claim 1, further comprising:
introducing a peroxide into the subsurface;
wherein the peroxide promotes proliferation of aerobic microorganisms and biodegradation of the biodegradable compound.

49. The method of claim 1, wherein the contaminant comprises a component selected from the group consisting of NAPL (non-aqueous phase liquid), DNAPL (dense non-aqueous phase liquid), LNAPL (light non-aqueous phase liquid), aromatic hydrocarbon, non-halogenated aromatic hydrocarbon, polyaromatic hydrocarbon, BTEX (benzene, toluene, ethyl benzene, and/or xylene), halogenated hydrocarbon, and combinations.

50. (canceled)

51. The method of claim 1, wherein the primary oxidant and the surfactant and/or cosolvent are simultaneously administered.

52. The method of claim 1, wherein the primary oxidant and the surfactant and/or cosolvent are sequentially administered.

53. The method of claim 1, wherein the primary oxidant oxidizing the solubilized contaminant and the microorganism biodegrading the biodegradable compound reduces the amount of contaminant in the subsurface to less than a predetermined level.

54. The method of claim 1, wherein the amount of residual primary oxidant remaining after oxidation of the solubilized contaminant is less than a predetermined level.

55. The method of claim 1, wherein the amount of residual surfactant and/or cosolvent remaining after oxidation of the solubilized contaminant is less than a predetermined level.

56. A method of designing a procedure for reducing the concentration of a contaminant at a site in a subsurface, comprising:
obtaining a sample representative of the contaminated site of interest;
testing the sample with various concentrations of primary oxidant, surfactant, and/or cosolvent under various conditions of temperature, pressure, and/or flow rate;
determining the rate of mobilization of the contaminant under the various concentrations and conditions;
determining the rate of biodegradation of the contaminant under the various concentrations and conditions; and
identifying an optimum set of concentrations and conditions for reducing the concentration of the contaminant at the site in the subsurface,
wherein the representative sample is selected from the group consisting of a core sample taken from the subsurface of the site and a simulated sample comprising soil similar to that of the subsurface of the site spiked with contaminant.

57. (canceled)

58. A system for reducing the concentration of a contaminant at a site in a subsurface, comprising:
an injection well;
an injection fluid injection system fluidly connected to the injection well;
an injection fluid comprising a primary oxidant and a surfactant and/or a cosolvent, the system being operable to promote biodegradation of the contaminant.

59. The system of claim 58, comprising:
a first pumping system that stores a primary oxidant and a surfactant and/or a cosolvent, mixes the primary oxidant and the surfactant and/or cosolvent in predetermined ratios, and injects the primary oxidant and the surfactant and/or cosolvent at a first injection point into an oxidation zone of the subsurface at a predetermined rate;
a second pumping system that stores a reducing agent and injects the reducing agent at a second injection point into a reducing zone of the subsurface at a predetermined rate;
a monitoring device that determines the concentration and/or spatial distribution in the subsurface of a quantity selected from the group consisting of contaminant, oxidant, surfactant, cosolvent, microorganisms, and combinations;
wherein the monitoring device can adjust the ratios of mixing the primary oxidant and the surfactant and/or cosolvent, the rate of injection of the primary oxidant and the surfactant and/or cosolvent, and the rate of injection of the reducing agent so as to maximize biodegradation by the microorganisms, minimize the concentration of the contaminant, minimize the time required to reduce the contaminant to a predetermined level, and/or minimize the amount of primary oxidant, surfactant, cosolvent, and/or reducing agent used.
